(19) United States
(12) Patent Application Publication
(10) Pub. No.: US 2016/0138019 A1
(43) Pub. Date: May 19, 2016

(54) Block Decoys

(71) Applicant: UCB BIOPHARMA SPRL, Brussels (BE)

(72) Inventors: Adam John Brown, Slough, Berkshire (GB); David Cameron James, Slough, Berkshire (GB)

(21) Appl. No.: 14/898,867

(22) PCT Filed: Jun. 17, 2014
(86) PCT No.: PCT/EP2014/062776

§ 371 Date: Dec. 16, 2015

(30) Foreign Application Priority Data
Jun. 18, 2013 (GB) 1310853.5

(51) Int. Cl. C12N 15/113; C12P 21/00

(52) U.S. Cl. CPC C12N 15/113; C12P 21/00; C12N 2310/13; C12N 2320/51; C12N 2310/532

(57) ABSTRACT

The present disclosure relates to a method of preparing exonuclease resistant molecules of block-decoys, use of the molecules in methods of modulating expression of recombinant proteins, particularly in vitro, for example by down regulation or inhibition of one or more transcription factors, and novel molecules of block-decoys, especially those obtained or obtainable from the methods herein. The disclosure also relates to use of said block decoys in vitro and in therapy. In one aspect there is provided a method for regulating recombinant gene expression in vitro comprising the steps of: a) providing a host cell encoding one or more recombinant genes for expression, b) contacting the cell with a exonuclease resistant block-decoy under condition suitable for the block-decoy to gain entry into the cell, and c) expressing the recombinant protein or protein.

The present disclosure relates to a method of preparing exonuclease resistant molecules of block-decoys, use of the molecules in methods of modulating expression of recombinant proteins, particularly in vitro, for example by down regulation or inhibition of one or more transcription factors, and novel molecules of block-decoys, especially those obtained or obtainable from the methods herein. The disclosure also relates to use of said block decoys, for example in vitro, ex vivo use and use in therapy.

Transcriptional output of a given gene at a specific time point is determined by the composition of transcription factor regulatory elements (TFREs) within its promoter and the availability of cognate transcription factors (TFs) within the cell (Coulon et al., 2013). Cellular transcriptomes are therefore a functional consequence of multiple TF-TFRE interactions occurring at thousands of discrete genomic loci. A mechanistic understanding of the TF-TFRE interactions regulating individual promoters' transcription would enable strategies to predictably control, manipulate and improve their activities. A mechanistic understanding of the TF-TFRE interactions regulating multiple promoters' activities within discrete pathways would enable strategies to engineer entire cellular processes. Characterisation of TF-TFRE interaction functionalities within the CHO cell factory could accordingly enable i) optimisation of product gene transcription rates throughout biomanufacturing processes and ii) cell line engineering strategies to achieve desirable bioproduction phenotypes, such as resistance to apoptosis and increased proliferation.

Physical disruption of TF binding to target sites is the most effective and well-established method of investigating TF-TFRE interactions. An effective method to achieve this is the use of transcription factor decoys (Tomita et al., 1999; Bezzerri et al., 2011; Renard et al., 2012); short synthetic oligodeoxynucleotides (ODN) that contain a specific TFRE motif. When introduced into a cell the decoys compete for available TFs, preventing their association at target promoters (Bielinska et al., 1990). This site-specific sequestration of TFs makes decoys an attractive method to determine the functional contribution of individual TFREs to a promoter's activity.

The key determinants of decoy effectiveness are stability, specificity, and uptake (Osako et al., 2012). Multiple methods of decoy formation have been developed to improve these factors, primarily focusing on their stability against intracellular nucleases. These include chemical modifications such as the use of phosphorothioate groups (Bielinska et al., 1990), and circular dumbbell ODNs that have enzymatically ligated termini (Osako et al., 2007), conferring resistance to exonucleases (the primary cause of intracellular degradation (Gamper et al., 1993)). Although such advancements have greatly improved decoy functionality, particularly in potential therapeutic applications (Gambari et al., 2011), currently available methods are not ideally suited to in vitro gene regulation studies.

As most promoters contain binding sites for multiple TFs, gene regulation studies utilising decoys are likely to require multiple decoys, targeting varying combinations of different TFREs. Ideally, where multiple TFREs are targeted at once they would be included on a single decoy molecule to avoid the uneven distribution of different decoys across the transfected cell population. Phosphorothioate and dumbbell decoys targeting two (Miyake et al., 2006; Lee et al., 2012) and three (Gao, 2006) TFREs have been described but these formation methods do not allow for the rapid creation of bespoke chimeric decoys. Further, they do not provide the capability to fine-tune the molar ratio of different sites within one molecule. Currently available tools are therefore poorly suited for in vitro investigations into multi-transcription factor mediated processes that may require multiple regulatory elements to be inhibited in varying combinations. Determination of the TF-TFRE interactions regulating promoters/cellular pathways in CHO cells is therefore intractable with current decoy methods.

The present method provides exonuclease resistant circular block decoy molecules which can be rapidly assembled from regulatory element blocks, to provide bespoke constructs for a specific application, wherein the ratio of regulatory element blocks can be varied as desired to render the molecule suitable for the intended purpose.

Thus in one aspect there is provided a method of preparing a circular double stranded block-decoy molecule resistant to exonucleases comprising:
a. forming doubled stranded regulatory element-blocks comprising a specific transcription factor binding site (regulatory element) by annealing complementary single stranded oligodeoxyribonucleic acids containing a motif to form said transcription factor binding site,
b. ligating the regulatory element-blocks formed in step a) to form a concatemer, and circularisation by ligation of the termini of concatemer formed in step b) to provide a block-decoy in the form of a circular molecule.

In one embodiment the regulatory element blocks employed in step a) have sticky ends.

Also provided is a circular double stranded exonuclease resistant block-decoy molecule comprising two or more regulatory element-blocks in tandem, for example comprising in the range of 2 to 30 regulatory element-blocks (for example 2 to 10 or 20), in particular wherein each regulatory element block is independently specific to a transcription factor.

In one embodiment the regulatory element-blocks are independently specific to a transcription factor selected from the group comprising nuclear factor kB response element, cyclic AMP response element and enhancer box.

In one embodiment molecules of the present disclosure and employed in the method herein comprise two or three different regulatory elements (i.e. regulatory elements directed to two or three different transcription factors). This is advantageous because it has been shown to the increase the inhibition of the decoys.

In one embodiment the block-decoy molecule is deoxyribonucleic acid.

In one embodiment the block-decoy molecule comprises 100 base pairs or more.

In one embodiment the molecules according to the disclosure are chimeric.

In one embodiment there is provided a method for regulating recombinant gene expression comprising the steps of:
a. providing a host cell comprising one or more recombinant genes for expression (for example encoding a protein or proteins of interest),
b. contacting the cell with a exonuclease resistant block-decoy under condition suitable for the block-decoy to gain entry into the cell, and
c. expressing the recombinant protein or proteins.

Step b) and c) may be performed concomitantly or sequentially, for example where step c) is performed after step b).

In one embodiment there is provided a method for regulating expression of a gene of interest in a host cell comprising the steps of:
a. providing a host cell comprising one or more genes of interest (for example encoding a protein or proteins of interest),
b. contacting the cell with a exonuclease resistant block-decoy under condition suitable for the block-decoy to gain entry into the cell, and
c. determining the expression level of the gene of interest, for example by measuring the level of expression of a protein encoded by the gene of interest, or by observing a particular change of phenotype or cell function or activity.

Step b) and c) may be performed concomitantly or sequentially, for example where step c) is performed after step b).

In one embodiment the method is an in vitro or in vivo method, for example an in vitro method.

The block-decoys and methods of the present disclosure have been shown by the present inventors to be effective in in vitro gene regulation and advantageously provide a mechanism for sophisticated levels of control of cellular transcription.

### BRIEF DESCRIPTION OF THE FIGURES

FIG. 1 Shows a schematic of block-decoy formation. (A) Single stranded oligonucleotides are annealed to form regulatory element-blocks containing a transcription factor binding site and a 4 bp single stranded overhang at 5′ termini. (B) Regulatory element-blocks are ligated together into extending concatamers which circularise (C), allowing intramolecular ligation of cohesive termini, yielding covalently closed circular block-decoys containing multiple copies of the target binding site (D).

FIG. 2 Shows circular block decoys contain numerous regulatory element binding sites. Agarose gel analysis of block decoys constructed from NFkB-RE (lanes A2,3), E-box (lanes A4,5) and CRE (lanes A6,7) regulatory element binding site blocks. Circularisation of a purified block decoy (B1) was demonstrated by (i) two further sequential ligation reactions (B2,3) which showed no additional increase in decoy size distribution and (ii) stability on digestion with Exonuclease III for 0, 1 and 6 h at 37° C. (lanes C2-C4) respectively. Lanes C5, 6 and 7 show digestion of linear DNA sampled at the same time points.

FIG. 3A Shows NFkB-RE block-decoys inhibit NFkB-RE mediated expression. CHO-S cells (2×10⁵) were co-transfected with a NFkB-RE-dependent GFP reporter plasmid with either a NFkB-RE block decoy (white bars) or a scrambled NFkB-RE block decoy (black bars) at concentration of 0.2-2 μg/ml DNA per transfection. GFP expression was quantified 24 h post-transfection Data were normalised with respect to GFP expression in the presence of the scrambled decoy in each case. Bars represent the mean+SEM of three independent experiments each performed in triplicate.

FIG. 3B+C C) Shows CHO-S cells (2×10⁵) were co-transfected with a NFkB-RE-dependent GFP reporter plasmid with 2 ug/ml scrambled NFkB-RE block decoy (control) or different regulatory element block decoys illustrating specific inhibition with NFkB-RE block decoy. B) Shows Co-transfection of NFkB-RE block-decoy (white bars) or scrambled NFkB-RE block decoy (black bars) and different GFP reporter plasmids varying in transcription factor specificity (CRE, E-box or NFkB-RE) illustrating specific inhibition of NFkB-RE mediated reporter expression. GFP expression was quantified 24 h post-transfection. Data were normalised with respect to GFP expression in the presence of the scrambled decoy in each case. In C and B each bar represents the mean+SEM of three independent experiments each performed in triplicate.

FIG. 3D Shows block-decoys inhibit NFkB-RE mediated expression throughout a four day GFP production process. CHO-S cells (2×10⁵) were co-transfected with a NFkB-RE-dependent GFP reporter plasmid with either a NFkB-RE block decoy (white bars) or a scrambled NFkB-RE block decoy (black bars) at concentration of 2 μg/ml DNA per transfection. GFP expression was quantified at varying timepoints post-transfection. Data were normalised with respect to GFP expression in the presence of the scrambled decoy in each case. Bars represents the mean+SEM of three independent experiments each performed in triplicate.

FIG. 4A Shows stoichiometric optimisation of chimeric block-decoys targeting multiple regulatory elements. In order to determine the correct stoichiometry of different TFRE-blocks in chimeric decoys to achieve equivalent inhibition of each regulatory element, the relative ability of separate TFRE-blocks to inhibit TFRE-specific reporter expression was first quantified. CHO-S cells were separately co-transfected with three different TFRE-specific block decoys (CRE, NFkB-RE and E-box) or the corresponding scrambled block-decoy controls at varying block decoy concentration with the corresponding TFRE-specific GFP-reporter plasmids (at a ratio of decoy:reporter plasmid maintained at 1:1). GFP expression in block-decoy transfected cells is shown as a percentage of reporter expression in cells transfected with the same concentration of scrambled decoy control. Best fit curves obtained by nonlinear regression analysis were utilised to determine the relative ratio of TFRE-specific blocks employed to construct chimeric decoys.

FIG. 5 A+B Shows chimeric block-decoys target multiple TFREs simultaneously. CHO-S cells (2×10⁵) were co-transfected with 3.5 chimeric block decoys and 2 μg/ml of either CRE, E-box or NFkB-RE SEAP-reporter plasmid Chimeric decoys were constructed using stoichiometric ratios of TFRE-blocks in the ratio A) NFkB-RE 1.0: E-box 0.62 and B) CRE 1.0: NFkB-RE 0.8: E-box 0.5 (control scrambled chimeric decoys contained the same ratio of scrambled TFRE-blocks). SEAP expression was quantified 24 h post-transfection. Each bar shows SEAP expression in chimeric decoy treated cells relative to expression with the same concentration of scrambled decoy. In A and B each bar represents the mean+SEM of three independent experiments performed in triplicate.

### DETAILED DESCRIPTION OF THE DISCLOSURE

As employed herein block-decoys are double stranded oligodeoxynucleotides (ODNs) useful as antigene strategies comprising two or more decoys or regulatory elements, for example in an exonuclease resistant structure, such as a circular molecule.

In one embodiment the two or more decoys are in tandem. That is to say in series one after the other in a continuous sequence, for example connected by short sequences of oligonucleotides, such as non-coding oligonucleotides.

Transcription factor decoys are a subgroup of decoys that is to say short, usually synthetic, oligodeoxynucleotides that sequester transcription factors and prevent their binding to targets in promoters (in particular the transcription factor regulatory elements associated therewith). In one embodiment the transcription factor decoys are single stranded. In one embodiment the transcription factor decoys are double stranded.

Oligodeoxynucleotides as employed herein refers to short polymers of single stranded deoxynucleotides including chemically modified versions thereof suitable for use in the annealing step of a method described herein. In one embodiment oligonucleotides are in the range 10 to 30 base pairs in length, for example 11, 12, 13, 14, 15, 16, 17, 18, 19, 20, 21, 22, 23, 24, 25, 26, 27, 28 or 29 base pairs.

Short oliogionucleotides as employed here in are in the range 10 to 30 base pairs in length, for example 11, 12, 13, 14, 15, 16, 17, 18, 19, 20, 21, 22, 23, 24, 25, 26, 27, 28 or 29 base pairs.

In one embodiment the oligodeoxynucleotides are synthetic, that is to say prepared by synthetic chemical techniques.

Regulatory element (also referred to herein as transcription factor regulatory element) as defined herein refers to a sequence of DNA that acts to regulate gene expression through the specific binding of transcription factors. Thus a regulatory element may also be a transcription factor decoy, in that when the motif of the transcription regulatory factor is reproduced and provided at a suitable concentration in the cell (i.e. not associated with the promoter or the gene for transcription) then these motifs compete with the transcription factor regulatory elements associated with the promoter to bind transcription factors.

Thus regulatory element-blocks as employed herein are double-stranded blocks typically containing a sticky end, for example as employed in the ligation step, which define a sequence of DNA that acts to regulate gene expression through the specific binding of transcription factors. Thus in one embodiment these regulatory element blocks contain a consensus sequence (also referred to herein as a motif) for a specific regulatory element that serves as the binding site for a specific transcription factor, or set of transcription factors, as appropriate.

Transcription factors are a group of proteins that bind to cis-regulatory DNA motifs (regulatory elements) within gene promoters and enhancers to regulate the levels of gene transcription. By binding to their specific cognate regulatory element, transcription factors can function to positively or negatively affect the rate of transcription, acting as activators or repressors. Transcription factors are modular proteins that contain a DNA-binding domain, which determines the specific regulatory element sequence that the transcription factor binds to, and a trans-activating domain. Once bound transcription factors can affect the transcriptional process by a variety of mechanisms, including; RNA polymerase recruitment/binding stability; histone/chromatin modifications; transcription initiation; escape from promoter-proximal pausing; nucleosome clearance; and transcription termination.

Block-decoys employ a transcription factor binding motif, which may include a consensus sequence, in the or each regulatory element. When introduced into cells the block decoy competes for the available transcription factor to which it is specific. If the block decoy binds the transcription factor it prevents association of the factor with the promoter.

Consensus sequence as employed herein refers to a sequence that is identical or similar to the sequence in a transcription factor regularly element, such that it binds the relevant transcription factor.

A regulatory element in the block decoy will generally be specific to a transcription factor, a non-exhaustive list of transcription factors includes: NFkB, CREB, and c-Myc, which bind respectively to the regulatory elements nuclear factor kB response element, cyclic AMP response element and enhancer box.

In one embodiment the block decoy according to the disclosure comprises or consist of regulatory elements to NFkB and CRE.

In one embodiment the block decoy according to the disclosure comprises or consists of a regulatory element directed to YY1. The latter may be useful in host cells expressing recombinant proteins because YY1 is thought by the present inventors to be a negative transcription factor for the CMV promoter.

Block-decoys can readily be designed to target any transcription factor for which a defined consensus binding site is known.

In one embodiment a block-decoy comprises a regulatory element specific to nuclear factor kB response element, for example the block-decoy may comprise or consist of a strand 5′-TCGATGGGACTTTCCA-3′ (SEQ ID NO: 1) and a complementary sequence 5′-TCGATGGAAAGTCCCA-3′ (SEQ ID NO: 2), wherein the consensus sequence is underlined.

In one embodiment a block-decoy comprises a regulatory element specific to cyclic AMP-response element, for example the block-decoy may comprise or consists of a strand 5′-TCGATTTGACGTCATT-3′ (SEQ ID NO: 3) and a complementary sequence 5′-TCGAAATGACGTCAAA-3′ (SEQ ID NO: 4), wherein the consensus sequence is underlined.

In one embodiment a block-decoy comprises a regulatory element specific to enhancer box, for example the block-decoy may comprise or consist of a strand 5′-TCGAAACACGTGAGA-3′ (SEQ ID NO: 5) and a complementary sequence 5′-TCGATCTCACGTGTT-3′(SEQ ID NO: 6), wherein the consensus sequence is underlined.

Other (non-exhaustive) examples of transcription factors that may be targeted by using the appropriate regulatory sequence indicated include: Activator protein 1, TGACTCA; CCAAT-enhancer Binding Protein α, TTGCGCAA; Cellular myeloblastosis protein, TAACGG; Elongation Factor 2, TTTCGCGC; Early Growth Response Protein 1, CGCCCCCGC; ERR-alpha, AGGTCATTTTGACCT (SEQ ID NO: 7); GATA-1, AGATAG; Growth Factor Independence 1, AAAATCAAC; Hepatocyte Nuclear Factor 1α, GGGCCAAAGGTCT (SEQ ID NO: 8); Insulin Promoter Factor 1, CCCATTAGGGAC (SEQ ID NO:9); IFN-stimulated gene factor 3, GAAAAGTGAAACC (SEQ ID NO: 10); Myocyte enhancer Factor 2, CTAAAAATAG (SEQ ID NO: 11); Nuclear Factor 1, TTGGCTATATGCCAA (SEQ ID NO: 12); Nuclear Factor of Activated T Cells, AGGAAATC; Octamer-1, ATTAGCAT; Retinoic Acid Receptor α, AGGTCATCAAGAGGTCA (SEQ ID NO: 13); and Specificity Protein 1, GGGGCGGGG; Yin Yang 1, CGCCATTTT.

| Transcription Factor | Sequence |
|---|---|
| Regulatory Element (RE) | |
| Activator protein 1 (AP1) | TGACTCA |
| CC(A/T)₆GG element (CArG) | CCAAATTTGG |
| SEQ ID NO: 14 | |
| CCAAT displacement protein | GGCCAATCT |
| (CDP) | |
| CCAAT-enhancer binding protein | TTGCGCAA |
| alpha (C/EBPα) | |
| Cellular myeloblastosis (cMyb) | TAACGG |
| cAMP RE (CRE) | TGACGTCA |
| Elongation factor 2 (E2F) | TTTCGCGC |
| E4F1 SEQ ID NO: 15 | GTGACGTAAC |
| Early growth response protein | CGCCCCCGC |
| 1 (EGR1) | |
| Estrogen-related receptor alpha | AGGTCATTTTGACCT |
| RE (ERRE) SEQ ID NO: 16 | |
| Enhancer box (E-box) | CACGTG |
| GATA-1 (GATA) | AGATAG |
| GC-box | GGGGCGGGG |
| Glucocorticoid RE (GRE) | AGAACATTTTGTTCT |
| SEQ ID NO: 17 | |
| Growth factor independence 1 | AAAATCAAC |
| (Gfi1) | |
| Helios RE (HRE) SEQ ID NO: 18 | AATAGGGACTT |
| Hepatocyte nuclear factor 1 | GGGCCAAAGGTCT |
| (HNF) SEQ ID NO: 19 | |
| Insulin promoter factor 1 | CCCATTAGGGAC |
| (IPF1) SEQ ID NO: 20 | |
| Interferon-stimulated RE | GAAAAGTGAAACC |
| (ISRE) SEQ ID NO: 21 | |
| Myocyte enhancer factor 2 | CTAAAAATAG |
| (MEF2) SEQ ID NO: 22 | |
| Msx homeobox (MSX) | CGGTAAATG |
| Nerve growth factor-induced | AAAGGTCA |
| gene-B RE | |
| Nuclear factor 1 (NF1) | TTGGCTATATGCCAA |
| SEQ ID NO: 23 | |
| Nuclear factor of activated | AGGAAATC |
| T cells (NFAT) | |
| Nuclear factor kappa B (NFκB) | GGGACTTTCC |
| SEQ ID NO: 24 | |
| Octamer motif (OCT) | ATTAGCAT |
| Retinoic acid RE (RARE) | AGGTCATCAAGAGGTCA |
| SEQ ID NO: 25 | |
| Yin yang 1 (YY1) | CGCCATTTT |
| Random 8 mer (8 mer) | TTTCTTTC |

Circular as employed herein refers to a molecule in the form of a circle. Plasmids are examples of circular molecules. Dumbbell shaped molecule comprising circular components and a linear component are not circular within the definitions as employed herein. To form a circle the molecules generally require about 100 base pairs or more. In one embodiment the circular molecules according to the disclosure comprise between approximately 90 and 350 base pairs, for example 100, 150, 200, 250 or 300, base pairs.

Exonuclease resistant as employed herein refers to substantially no change in size of the molecule when incubated with an exonuclease, such as Exonuclease III, for example for a period of 1 to 24 hours, such as 2, 3, 4, 5, 6, 7, 8, 9, 10, 11, 12, 13, 14, 15, 16, 17, 18, 19, 20, 21, 22 or 23 hours. Change is size can be measured by know techniques, for example as described in the examples. Substantially no change in size of the molecules as employed herein includes a population of molecules where an insignificant number of molecules are digested by the exonuclease, for example 5% or less, such as 4, 3, 2, 1% or less which.

Concatemer as employed herein refers to a continuous linear double stranded deoxyribonucleic acid molecule comprising multiple oligodeoxyribonucleic acids of regulatory element sequences in series (tandem) thereby forming a linear block decoy molecule. Generally a concatemer will require in the region of about 100 base pairs to allow the termini to be ligated to form a circular molecule according to the disclosure.

Double stranded as employed herein refers to two strands of sequences associated together for example strands of oligodeoxyribonucleic acid which are complementary.

Complementary as employed herein refers to base pairing in nucleotides, for example thymine being paired with adenine and cytosine paired with guanine.

Molecule as employed herein refers to the assembled or partly assembled constructs of the disclosure, as appropriate in the context.

Where combinations of regulatory element-blocks are incorporated into a single block-decoy molecule the relevant ratios of the regulatory element-blocks employed can be arranged according to a given design to provide the requisite level of control over gene expression.

Where multiple transcription factors are to be targeted simultaneously by a combination of regulator elements each of which is specific to a particular transcription factor it is advantageous to incorporate all of the specific regulatory elements (cognate regulator elements) into a single block decoy molecule as this may minimise inter-experiment/process variability, particularly by avoiding the uneven distribution of different decoys across the transfected cell population, and thus provide a more robust product to work with.

Chimeric in the context of block decoy molecules refers to where the molecule comprises at least two regulatory elements, wherein one regulatory element is specific to a first transcription factor and the second regulatory element is specific to a different transcription factor.

In one embodiment the molecules herein comprise in the range of 2 to 30 regulator element-block or 2 to 30 copies of a regulatory element-block. Thus the regulatory blocks can be the same or different sequences and/or specificities.

In one embodiment the molecule according to the disclosure comprise in the range of 5 to 30 (such a 6, 7, 8, 9, 10, 11, 12, 13, 14, 15, 16, 17, 18, 19, 20, 21, 22, 23, 24, 25, 26, 27, 28 or 29 regulatory element blocks in total, such as 7 to 27 regulatory element blocks. Thus in one embodiment there are multiple copies of one or more RE-blocks in molecules of the present disclosure.

Where a small number of regulatory element-blocks are employed then additional base pairs may be required to prepare a concatemer of appropriate length to allow circularisation. Suitable base pairs may be provided in the form of dummy regulator element blocks which are essentially scrambled regulatory element-blocks which do not bind transcription factors. The present inventors have shown that such sequences do not interfere with the activity of the block decoy. These blocks may be provided at one or both ends of the regulatory element blocks, and/or there between.

The molecules of the present disclosure can be prepared by first annealing complementary single stranded oligodeoxyribonucleic acid molecules containing a binding motif (such as a transcription factor motif) under suitable conditions to provide double stranded units, preferably with sticky ends.

Suitable conditions for annealing includes an initial denaturation step performed at an elevated temperature, for example heating at 90-99° C. for a suitable period, such as less than 10 minutes, in particular about 5 mins, followed by an annealing step where the reaction mixture is ramp cooled at an appropriate rate of temperature decrease to an appropriate temperature, for example decreasing the temperature by 0.5-1.5° C./min to a final temperature of 4-27° C.

Sticky ends as employed herein refers to where the double stranded sequences having one strand with overhanging base pairs, for example about 3 and 12 additional base pairs, such as 4, 5, 6, 7, 8, 9, 10 or 11 overhanging base pairs, such as 4 overhanging base pairs.

These sticky ends are complementary to overhanging base pairs in other units to assist the ligation at the next stage.

The sticky ends may be the 5′ end, or the 3′ end. In one embodiment the 5′ ends are sticky.

In one embodiment single stranded oligodeoxynucleotides (employed to the form the regulatory elements) are phosphorylated at the 5′ terminus, such that regulatory element-blocks contain a phosphate group at the 5′ terminus of both strands. This allows regulatory element-blocks to be ligated together via the formation of phosphodiester bonds.

Ligation of the double stranded regulatory element-blocks will generally be effected employing a suitable enzyme, for example a ligase (EC 6.5.1.1), such as T4 DNA ligase, under appropriate conditions, for example 20-30° C., such as 21, 22, 23, 24, 25, 26, 27, 28 or 29° C. and for a suitable period such as 2 to 5 hours, in particular 3 or 4 hours.

In one embodiment 5 units or more of high concentration enzyme are employed, where one ligation unit catalyses the exchange of 1 nmol ³²P-labelled pyrophosphate into ATP in 20 min at 37° C. This is equivalent to approximately 300 cohesive-end ligation units, where one cohesive-end ligation unit is the amount required to give 50% ligation of Hind III fragments of lambda DNA in 30 min at 16° C.

The double stranded regulatory element-blocks are ligated to form a concatemer, for example of about 100 base pairs, after which the molecule is flexible and the termini can ligate to form a circular molecule of the disclosure. Generally the concatemer will not be isolated and no separate step is required to cause the termini to ligate and form a circular molecule.

In one embodiment there is provided a circular double stranded deoxyribonucleic molecule obtained or obtainable from said method.

The method is advantageous in that it allows the rapid assembly of an exonuclease resistant block-decoy molecule allowing bespoke constructs to be tailored to a given situation and the preparation of a library of such molecules providing a repertoire of specificities to improve the likelihood that one will suit a given situation. It also allows the mechanisms of transcription factors to be investigated in a systematic way.

In one embodiment the library comprises between 10 and 1000 molecules.

In one example, the block-decoys of the present invention may be used to investigate any multi-transcription factor mediated cell function or phenotype in vitro or in vivo.

The block decoy molecules of the present disclosure may be useful in therapeutic applications and there is provided use of a block decoy molecule described herein in therapy.

In one embodiment the block decoy element described herein are provided on a plasmid. A plasmid as employed herein a circular day able to replicate in suitable cells, and comprise an origin of replication, optionally a marker such as antibiotic resistant gene and optionally a promoter for replicating the oliogonucleotides encoded by the plasmid. Also provided is use of the plasmid in accordance with the disclosure herein, in particular the method(s) described herein.

In one embodiment the block decoy molecule of the present disclosure is suitable for (or employed for) controlling the expression of recombinant genes in vitro, in particular genes encoding proteins, such as therapeutic proteins including antibodies and binding fragments thereof.

Thus in one embodiment there is provided a method for regulating recombinant gene expression in vitro comprising the steps of:
a. providing a host cell encoding one or more recombinant genes for expression,
b. contacting the cell with a exonuclease resistant block-decoy under condition suitable for the block-decoy to gain entry into the cell, and
c. expressing the recombinant protein or proteins, and
d. optionally further comprises the step of recovering the protein or proteins.

In one embodiment the block decoy molecule of the present disclosure is suitable for (or employed for) altering or regulating any multi-transcription factor mediated cell function or phenotype in vitro or in vivo.

In one embodiment there is provided a method for regulating expression of a gene of interest in a host cell comprising the steps of:
a. providing a host cell comprising one or more genes of interest (for example encoding a protein or proteins of interest),
b. contacting the cell with a exonuclease resistant block-decoy under condition suitable for the block-decoy to gain entry into the cell, and optionally
c. determining the expression level of the gene of interest, for example by measuring the level of expression of a protein encoded by the gene of interest, or by observing a particular change of phenotype or cell function or activity.

Conditions suitable for the block decoy to gain entry into the host cell include known transfection methods, including chemical transfection methods such as lipofection, and non-chemical techniques such as electroporation and the like. In one embodiment the transfection employed in step b) is lipofection.

Recombinant gene as employed herein refers to a gene which is not natural to the cell and is introduced transiently or stably into the cell by recombinant techniques.

In one embodiment the protein is an antibody or binding fragment thereof.

The term ‘antibody’ as used herein generally relates to intact (whole) antibodies i.e. comprising the elements of two heavy chains and two light chains. The antibody may comprise further binding domains for example as per the molecule DVD-Ig as disclosed in WO 2007/024715, or the so-called (FabFv)₂Fc described in WO2011/030107. Thus antibody as employed herein includes bi, tri or tetra-valent antibodies.

Binding fragments of antibodies include single chain antibodies (i.e. a full length heavy chain and light chain); Fab, modified Fab, Fab′, modified Fab′, F(ab′)2, Fv, Fab-Fv, Fab-dsFv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217), for example the FabFv formats disclosed in WO2009/040562 and disulphide stabilised versions thereof as disclosed in WO2010/035012. The methods for creating and manufacturing these antibody fragments are well known in the art (see for example Verma et al., 1998, Journal of Immunological Methods, 216, 165-181). Other antibody fragments for use in the present invention include the Fab and Fab′ fragments described in WO2005/003169, WO2005/003170 and WO2005/003171. Multi-valent antibodies may comprise multiple specificities e.g. bispecific or may be monospecific (see for example WO 92/22853 and WO05/113605).

Typical Fab′ molecule comprises a heavy and a light chain pair in which the heavy chain comprises a variable region V_{H}, a constant domain C_{H}1 and a hinge region and the light chain comprises a variable region V_{L} and a constant domain C_{L}.

In one embodiment there is provided a dimer of Fab′ according to the present disclosure for example dimerisation may be through the hinge.

In one embodiment the antibody or binding fragment is human or humanised

As used herein, the term ‘humanised antibody molecule’ refers to an antibody molecule wherein the heavy and/or light chain contains one or more CDRs (including, if desired, one or more modified CDRs) from a donor antibody (e.g. a non-human antibody such as a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment only the specificity determining residues from each of the CDRs described herein above are transferred to the human antibody framework.

In one embodiment the host cells is a prokaryotic cell or eukaryotic cell, for example bacterial cell such as E. coli an insect cell, or a mammalian cell, for example CHO cell, HEK cell or similar. In one embodiment the cell is a CHO-S or CHO-K1 cell or a derivative thereof. A derivative thereof is a cell obtained or adapted from said cell.

In one embodiment the block-decoy molecules are according to the present disclosure target transcription factors which regulate the CMV promoter.

In one embodiment there is provided a protein prepared by the method.

In vitro as employed herein simply means an method performed in the laboratory in “glass” and not in vivo.

In the context of this specification “comprising” is to be interpreted as “including”.

Aspects of the invention comprising certain elements are also intended to extend to alternative embodiments “consisting” or “consisting essentially” of the relevant elements.

Embodiment herein may be combined where technical appropriate.

Aspects or embodiments described herein may be employed as basis for a negative disclaimer.

The disclosure will now be illustrated by reference to the following non-limiting examples.

### Examples

Acronyms and Abbreviations

| | |
|---|---|
| AP1 | Activator protein 1 |
| BRE | TFIIB recognition elements |
| C/EBPα | CCAAT-enhancer binding protein alpha |
| CArG | CC(A/T)₆GG element |
| CDP | CCAAT displacement protein |
| CHEF-1α | Chinese hamster elongation factor-1α |
| CHO | Chinese hamster ovary |
| cMyb | cellular myeloblastosis |
| CPRE | core promoter regulatory element |
| CRE | cyclic adenosine monophosphate-RE |
| CRM | cis-regulatory module |
| DCE | downstream core element |
| DHFR | Dihydrofolate reductase |
| DPE | downstream promoter element |
| DTE | difficult-to-express |
| E-box | Enhancer box |
| E2F | Elongation factor 2 |
| EGR1 | Early growth response protein 1 |
| ERRE | Estrogen-related receptor alpha-RE |
| FACS | fluorescence-activated cell sorting |
| Gfi | Growth factor independence |
| GFP | Green fluorescent protein |
| GRE | glucocorticoid-RE |
| GS | Glutamine synthetase |
| hCMV-IE1 | human cytomegalovirus immediate early one |
| HDAC | histone deacetylase |
| HNF | hepatocyte nuclear factor |
| HRE | helios-RE |
| Inr | initiator element |
| IPF | insulin promoter factor |
| ISRE | interferon-stimulated-RE |
| IVCD | integral of viable cell density |
| KO | knockout |
| LTR | long terminal repeat |
| mAb | monoclonal antibody |
| MAR | matrix attachment region |
| MCS | multiple cloning site |
| MEF | myocyte enhancer factor |
| MPRA | massively parallel reporter assay |
| MSX | msx homeobox |
| MTE | motif 10 element |
| NBRE | nerve growth factor-induced gene-B-RE |
| NF1 | nuclear factor 1 |
| NFAT | nuclear factor of activated T cells |
| NFκB | nuclear factor kappa B |
| OCT | octamer motif |
| ODN | oligodeoxynucleotide |
| ORF | open reading frame |
| PIC | preinitiation complex |
| PTM | post translational modification |
| RARE | retinoic acid-RE |
| RE | regulatory element |
| RMCE | recombination mediated cassette exchange |
| SEAP | Secreted alkaline phosphatase |
| siRNA | short interfering RNA |
| SOI | site of integration |
| SV40E | simian virus 40 early promoter and enhancer |
| TESS | Transcription Element Search System |
| TF | transcription factor |
| TFRE | transcription factor regulatory element |
| TGE | transient gene expression |
| TRAP | Transcription Affinity Prediction tool |
| TSS | transcriptional start site |
| UR | unique region |
| UTR | untranslated region |
| XCPE | X core promoter element |
| YY1 | Yin yang 1 |

Construction of Block-Decoys

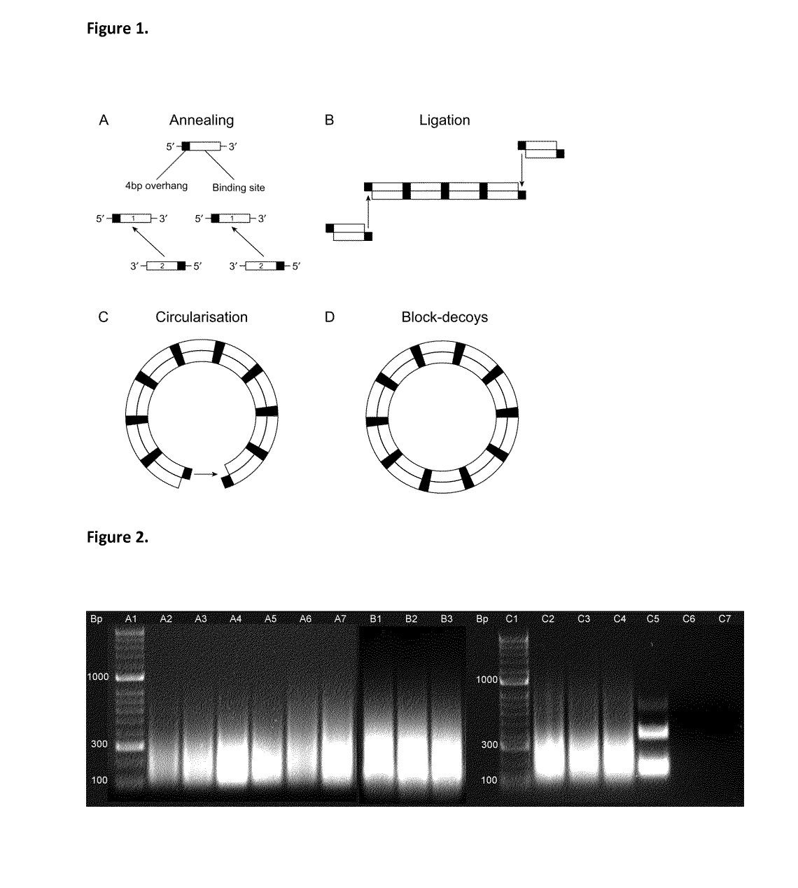

The method of block-decoy construction is shown schematically in FIG. 1. Regulatory element (RE)-block molecules were developed by annealing two complementary, single stranded 5′ phosphorylated DNA ODNs (Sigma, Poole, UK) in STE buffer (100 mM NaCl, 50 mM Tris-HCl, 1 mM EDTA, pH 7.8, Sigma). ODNs were heated at 95° C. for 5 min and then ramp cooled to 25° C. over 2 h. RE-blocks (12 μg) were then ligated with 5 units of high concentration T4 DNA ligase (Life Technologies, Paisley, UK) at room temperature for 3 h to create RE-blocks that contain a transcription factor binding site and a 4 bp TCGA single stranded overhang at each 5′ termini. The cohesive ends enable RE-blocks to be ligated together into extending concatamers. At sizes greater than 100 bp DNA molecules are likely to bend (13, 14), allowing ligation of cohesive termini (15, 16). Therefore, ligation of input blocks theoretically results in covalently closed circular block-decoys. Chimeric decoys were constructed by ligating varying molar concentrations of different RE-blocks. The sequences of RE-blocks employed were as follows (consensus site underlined): nuclear factor kB response element (NFkB-RE), 5′-TCGATGGGACTTTCCA-3′ and 5′-TCGATGGAAAGTCCCA-3′; cyclic AMP-responsive element (CRE), 5′-TCGATTTGACGTCATT-3′ and 5′-TCGAAATGACGTCAAA-3′; Enhancer box (E-box), 5′-TCGAAACACGTGAGA-3′ and 5′-TCGATCTCACGTGTT-3′. Scrambled decoys contained the following scrambled consensus sites: NFkB-RE, AATCGCAAGT; CRE, GACTAGAG; E-box, GCTCAG. All block-decoys were extracted and stored at 350 ng/μl.

Analysis of Block Decoy Structure

Block-decoy population size distribution was analyzed by ethidium bromide agarose gel electrophoresis utilizing molecular weight markers (Hyperladder II, Bioline, London, UK). To confirm block-decoys circularization, 1.5 μg of purified block-decoy was added to 5 units of high concentration T4 DNA ligase before gel analysis. To test the stability of block-decoys against exonuclease, 4 μg of block-decoy was incubated with 300 units of Exonuclease III (Promega, Southampton, UK) and the mixture was incubated at 37° C. A mixture of linear ODNs spanning the molecular weight range of the block-decoys was used as a positive control.

Construction of RE-Specific Reporter Vectors

A promoterless vector was subcloned from pSEAP2control (Clontech, Oxford, UK) by PCR amplification of appropriate vector regions with Phusion high fidelity polymerase (New England Biolabs, Hitchin, UK). A minimal core promoter from the human Cytomegalovirus (CMV) was synthesized (Sigma) and cloned into the XhoI and EcoRI sites directly upstream of the secreted alkaline phosphatase (SEAP) open reading frame (ORF). The core promoter sequence used was as follows: 5′-AGGTCTATATAAGCAGAGCTCGTTTAGTGAACCGTCAGATCGCCTAGATACGCCATCCAC GCTGTTTTGACCTCCATAGAAGAC-3′. A second reporter plasmid was created by replacing the SEAP ORF with the turbo green fluorescent protein (GFP) ORF. To create binding site reporter plasmids, synthetic oligonucleotides containing 7× repeat copies of NFkB-RE, CRE and E-box were synthesized (Sigma), PCR amplified, and inserted into KpnI and XhoI sites upstream of the CMV core promoter. The sequence of all plasmid constructs was confirmed by DNA sequencing.

Cell Culture and Transfection

CHO-S cells, a suspension adapted variant of CHO-K1, were obtained from Life Technologies. CHO-S cells were routinely cultured in CD-CHO medium (Life Technologies) supplemented with 8 mM L-glutamine (Sigma) at 37° C. in 5% (v/v) CO₂ in vented Erlenmeyer flasks (Corning, UK), shaking at 140 rpm. Cells were subcultured every 3-4 days at a seeding density of 2×10⁵ cells/ml. Cell concentration and viability were determined by an automated Trypan Blue exclusion assay using a Vi-Cell cell viability analyser (Beckman-Coulter, High Wycombe, UK). Two hours prior to transfection, 2×10⁵ cells from a mid-exponential phase culture were seeded into individual wells of a 24 well plate (Nunc, UK). Cells were transfected with DNA-lipid complexes comprising 1 μg DNA per 3 μl Lipofectamine (Life Technologies), prepared according to the manufacturer's instructions. Transfected cells were incubated for 24 h prior to protein expression analysis. All transfections were carried out in triplicate and experiments repeated three times.

Quantification of Reporter Expression

SEAP protein expression was quantified using the Sensolyte pNPP SEAP colorimetric reporter gene assay kit (Cambridge Biosciences, Cambridge, UK) according to the manufacturer's instructions. GFP protein expression was quantified using a Flouroskan Ascent FL Flourometer (Excitation filter: 485 nm, Emission filter: 520 nm). Background fluorescence/absorbance was determined in cells transfected with a promoterless vector.

Results and Discussion

Block-Decoy Formation and Stability

Block-decoy formation (FIG. 1) was confirmed by gel electrophoresis. This analysis showed that different RE-specific decoys constructed using the appropriate RE-blocks exhibited near-identical size distributions, with the vast majority of molecules between 100-300 bp in size (FIG. 2). To test the hypothesis that circularization of decoys prevented further ligation (thus limiting their size) we (i) utilized purified block-decoys as the substrate in further ligation reactions and (ii) evaluated block-decoy stability against digestion by Exonuclease III active against linear DNA. In both cases, no variation in block-decoy size distribution was observed (FIG. 2). We conclude that this method of block-decoy construction yielded circular ODN containing approximately 7-20 copies of a target TF binding site.

Block-Decoy Function and Specificity

The use of block-decoys to inhibit the activity of specific regulatory elements was evaluated using GFP and SEAP reporter plasmids containing 5-7 copies of a discrete RE motif upstream of a core hCMV promoter. Preliminary experiments showed that minimal reporter expression was observed with the core promoter alone (1-3% of reporter activity of RE-containing plasmids; data not shown).

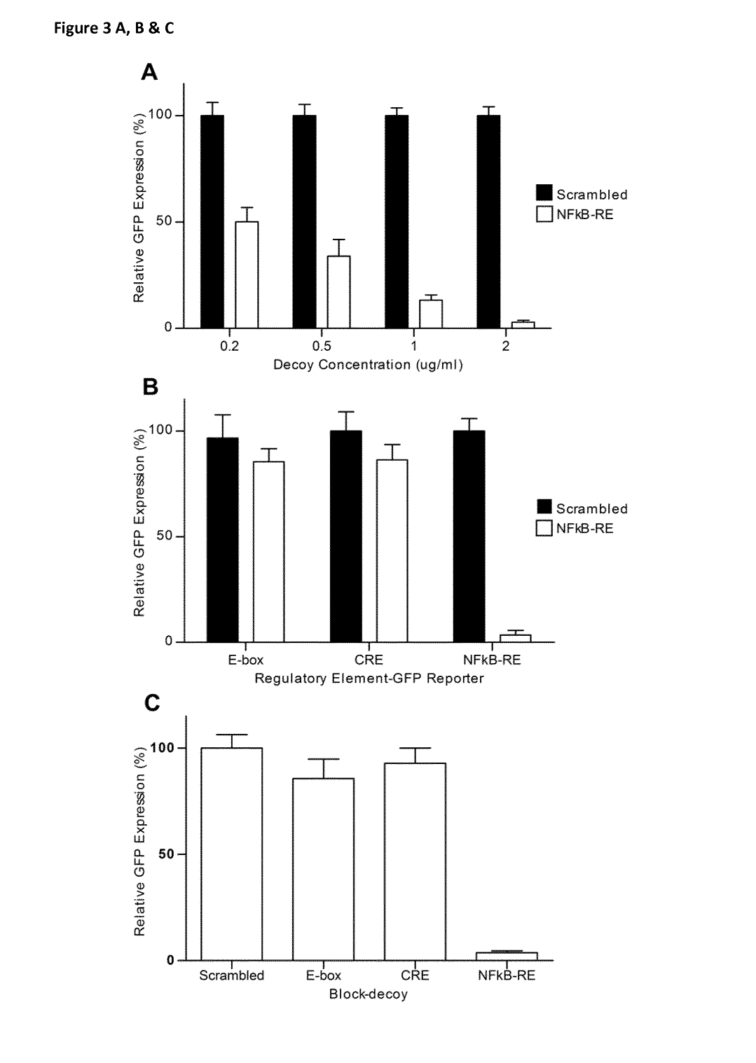

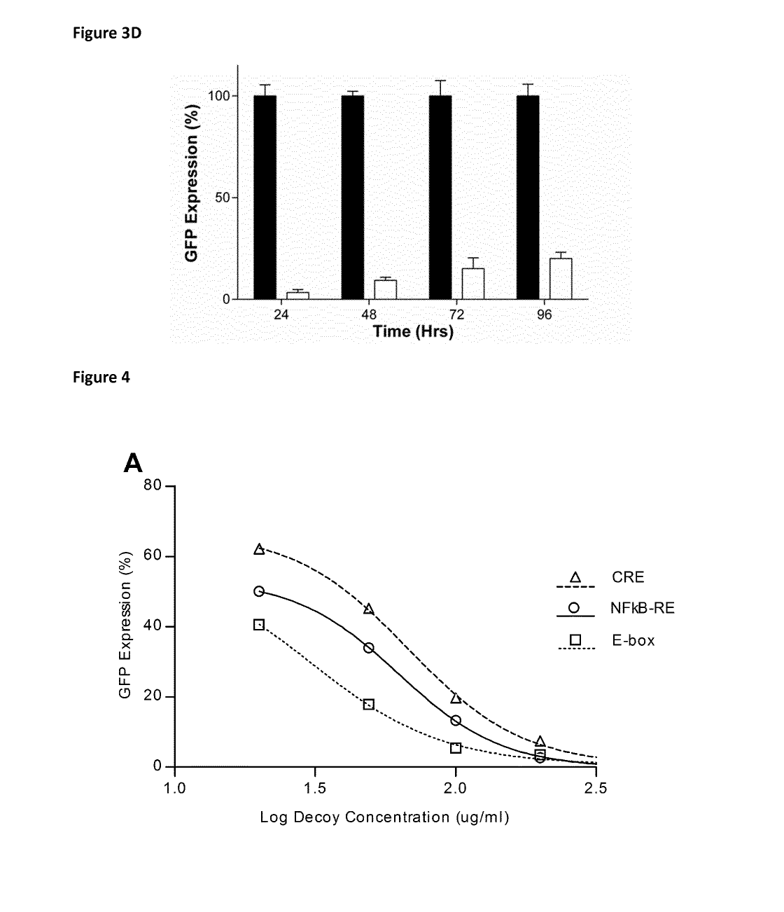

Three reporter plasmids, each utilizing specific REs (NFKB-RE, CRE or E-box) to drive reporter expression were used to validate the specific inhibitory effects of block-decoys in vitro. In each case, RE-specific reporter expression was inhibited only by the corresponding block-decoy. For example, as shown in FIG. 3A, the NFkB-RE block-decoy inhibited expression from NFkB-RE-GFP reporter plasmid in a dose-dependent manner. Moreover, the concentration of NFkB-RE block-decoy exhibiting maximal inhibition of NFkB-RE-GFP reporter expression (2 μg/ml) had no significant effect on GFP expression from either CRE-RE or E-box-RE reporter plasmids (FIG. 3B). Lastly, block-decoys constructed from E-box and CRE RE-blocks did not significantly affect expression from NFkB-RE-GFP reporter plasmid (FIG. 3C). All block-decoys exhibited similar RE-specific inhibition (data not shown), and we conclude that each block-decoy functions to specifically sequester its cognate regulatory element-binding transcription factors, inhibiting expression from promoters dependent on their activity.

Chimeric Block-Decoys

A major advantage of the block-decoy strategy is that it can be utilized to construct stoichiometrically optimized chimeric decoys targeting multiple REs. To demonstrate this novel capability we constructed a chimeric block decoy targeting all three REs; NFKB-RE, CRE and E-box.

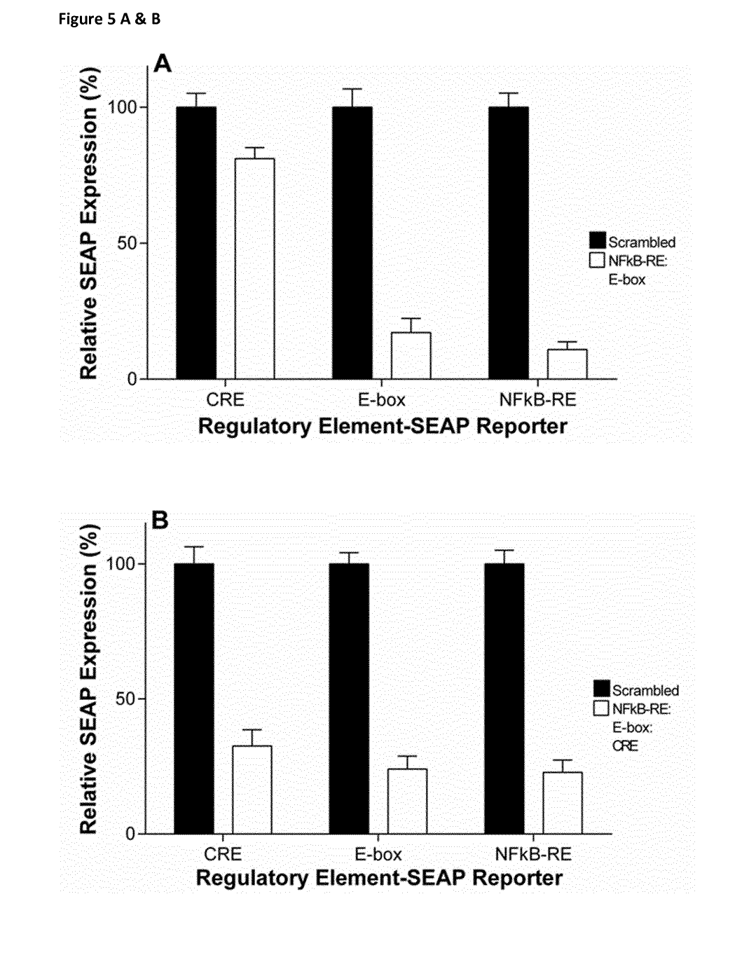

In order to determine the optimal ratio of RE-blocks to construct a chimeric block decoy exhibiting maximal, equivalent inhibition of all RE-reporter plasmids we adjusted the stoichiometry of RE-blocks in the ligation reaction according to the extent individual RE-specific block-decoys inhibited expression of the cognate RE-reporter (i.e. the relative ‘potency’ of each RE-block). We assume that the relative extent to which each RE-specific block-decoy inhibits reporter expression from its corresponding RE-reporter plasmid is a function of block-decoy specific differences in (i) the relative intracellular abundance of TFs and (ii) TF-RE block binding kinetics. As shown in FIG. 4A each RE-specific block decoy exhibited a characteristic inhibitory dose-response relationship, where at the highest concentrations expression from each corresponding RE-specific reporter was inhibited over 90%. Log transformation of block-decoy concentration data and nonlinear regression analysis enabled determination of the relative potency of each block-decoy, and revealed that their inhibitory potency occurred in the order: E-box>NFKB-RE>CRE, with a stoichiometry of E-box: 0.5: NFkB-RE: 0.8: CRE: 1.0 (calculated by interpolation to determine relative inhibitory concentrations). Thus, to achieve concurrent inhibition of all REs to a similar extent using the block-decoy approach we ligated RE-blocks in this stoichiometric molar ratio.

Anticipating that chimeric decoys would require a greater concentration of decoy to be transfected to achieve a specific reduction in RE-reporter expression (as the number of copies of each RE-block is effectively reduced with an increase in the number of different RE-blocks utilized to construct a chimeric decoy) we (i) increased total RE-decoy DNA load per transfection and (ii) utilized alternative RE-SEAP reporter constructs to enable more sensitive detection of RE driven reporter expression. Preliminary experiments showed that a decoy concentration of 3.5 μg/ml decoy was the maximal decoy load that could be co-transfected with RE-reporter plasmid whilst still maintaining quantitation in the linear range from each RE-specific SEAP reporter plasmid (transfected at 2 μg/ml) (data not shown). Chimeric decoys were therefore transfected at this concentration, equating to RE-block concentrations of 0.76 (E-box), 1.22 (NFkB-RE) and 1.52 μg/ml (CRE). Through interpolation of the single decoy data summarized in FIG. 4A we predicted that under these conditions chimeric decoys would inhibit expression from E-box, NFkB and CRE-SEAP reporter plasmids by 88%.

FIG. 4B shows that the chimeric decoy significantly inhibited expression from all three REs at approximately equivalent levels. E-Box, NFkB-RE and CRE dependent SEAP expression was reduced to 77%, 76% and 68% respectively, showing the chimeric decoy simultaneously sequestered a substantial proportion of the intracellular cognate TFs that bind to each of the three REs. The slight reduction in decoy potency compared to predicted values may be explained by (i) the reduced transfection efficiency associated with transfecting a higher concentration of DNA (resulting in less copies of each RE-block per cell) or (ii) TF-binding dynamics being affected by the presence of multiple RE-blocks. Nonetheless, the results show that three transcription factor binding pathways can be inhibited simultaneously using a chimeric block-decoy containing stoichiometrically tailored quantities of each RE-block. This is the first time that a transcription factor decoy has been shown to target multiple elements by using an optimised number of copies of each binding site.

It is likely that greater concentrations of chimeric decoy would have increased inhibition of each target element. In these experiments the concentration of block-decoy was limited by co-transfection with binding-site reporters. Promoters investigated in vitro are commonly either endogenous or significantly stronger than single RE promoters. In these cases higher decoys concentrations could be employed. Nonetheless, when DNA load is a restricting factor in vitro, block-decoys offer a significant advantage for concurrent inhibition of multiple REs. Fine-tuning of binding site copy ratios reduces the final decoy concentration required. This potentially enables a greater number of elements to be targeted simultaneously compared to existing methods. Adjustable control of RE-block ratios enables the optimal inhibition of each target element at any decoy concentration.

The results show block-decoys are a powerful tool for inhibiting multiple REs in vitro. The method's primary advantages are the ability to rapidly construct chimeric molecules and to control their binding site ratios. However, block-decoys have other potential benefits. Circular DNA has been associated with improved transfection efficiencies, compared to linear ODN (17, 18). Further, multiple copies of the same binding site within a single decoy molecule may enhance TF sequestration (19, 20). It was previously shown that decoys with three site copies achieved stronger inhibition than those containing a single site (12). Therefore, the 7-20+ binding sites per block-decoy may enhance decoy function and efficiency.

If a promoter of interest contains eight unique binding sites there are over 200 possible unique chimeric site combinations. Using the block-decoy methodology any of these could be constructed rapidly following the initial synthesis of eight RE-blocks. This compares to existing methods, where all chimeras would have to be synthesised independently. By utilising block-decoys the binding site ratios within each chimera could be adjusted to precisely control the relative extent to which each RE is inhibited in each transfected cell. This is a major advantage over the use of mixtures of single decoys, whose relative distribution within transfected cells is unpredictable. Current methods do not allow this sophisticated control of TF activity. Block-decoys therefore offer significant potential benefits for in vitro gene regulation studies.

### CONCLUSION

Block-decoys are a novel method of transcription factor decoy formation. The method described enables construction of chimeric decoys containing stoichiometrically optimised ratios of input RE-blocks. We demonstrated that block-decoys are able to inhibit expression from multiple target elements simultaneously using a bespoke chimeric ODN. Block-decoys have significant advantages over existing decoy methods for studies requiring the simultaneous inhibition of multiple elements in defined combinations. Block-decoys could be applied to the investigation of any multi-transcription factor mediated cell function or phenotype.

### REFERENCES

[1] N. J. Fuda, M. B. Ardehali, J. T. Lis, Defining mechanisms that regulate RNA polymerase II transcription in vivo. Nature 461 (2009) 186-192.
[2] S. Tomita, N. Tomita, T. Yamada, L. Zhang, Y. Kaneda, R. Morishita, T. Ogihara, V. J. Dzau, M. Horiuchi, Transcription factor decoy to study the molecular mechanism of negative regulation of renin gene expression in the liver in vivo. Circ. Res. 84 (1999) 1059-1066.
[3] V. Bezzerri, M. Borgatti, A. Finotti, A. Tamanini, R. Gambari, G. Cabrini, Mapping the transcriptional machinery of the IL-8 gene in human bronchial epithelial cells. J. Immunol 187 (2011) 6069-6081.
[4] E. Renard, B. Porée, C. Chadjichristos, M. Kypriotou, L. Maneix, N. Bigot, F. Legendre, D. Ollitrault, B. De Crombrugghe, F. Malléin-Gérin, Sox9/Sox6 and Sp1 are involved in the insulin-like growth factor-I-mediated upregulation of human type II collagen gene expression in articular chondrocytes. J. Mol. Med 90 (2012) 649-666.
[5] A. Bielinska, R. A. Shivdasani, L. Zhang, G. J. Nabel, Regulation of gene expression with double-stranded phosphorothioate oligonucleotides. Science 250 (1990) 997-1000.
[6] M. Kiomy Osako, H. Nakagami, R. Morishita, Modification of Decoy Oligodeoxynucleotides to Achieve Stability and Therapeutic Efficacy. Curr. Top. Med. Chem. 12 (2012) 1603-1607.
[7] M. K. Osako, N. Tomita, H. Nakagami, Y. Kunugiza, M. Yoshino, K. Yuyama, T. Tomita, H. Yoshikawa, T. Ogihara, R. Morishita, Increase in nuclease resistance and incorporation of NF-κB decoy oligodeoxynucleotides by modification of the 3′-terminus. J. Gene. Med 9 (2007) 812-819.
[8] H. B. Gamper, M. W. Reed, T. Cox, J. S. Virosco, A. D. Adams, A. A. Gall, J. K. Scholler, R. B. Meyer, Facile preparation of nuclease resistant 3′ modified oligodeoxynucleotides. Nucleic Acids Res. 21 (1993) 145-150.
[9] R. Gambari, Recent patents on therapeutic applications of the transcription factor decoy approach. Expert Opin. Ther. Pat. 21 (2011) 1755-1771.
[10] T. Miyake, M. Aoki, H. Nakashima, T. Kawasaki, M. Oishi, K. Kataoka, K. Tanemoto, T. Ogihara, Y. Kaneda, R. Morishita, Prevention of abdominal aortic aneurysms by simultaneous inhibition of NFKB and ets using chimeric decoy oligonucleotides in a rabbit model. Gene Ther. 13 (2006) 695-704.
[11] W. R. Lee, K. H. Kim, H. J. An, Y. Y. Park, K. S. Kim, C. K. Lee, B. K. Min, K. k. Park, Effects of Chimeric Decoy Oligodeoxynucleotide in the Regulation of Transcription Factors NF-κB and Sp1 in an Animal Model of Atherosclerosis. Basic Clin. Pharmacol. Toxicol 112(4) (2012) 236-243.
[12] H. Gao, J. Xiao, Q. Sun, H. Lin, Y. Bai, L. Yang, B. Yang, H. Wang, Z. Wang, A single decoy oligodeoxynucleotide targeting multiple oncoproteins produces strong anticancer effects. Mol. Pharmacol. 70 (2006) 1621-1629.
[13] Y. Zhang, D. M. Crothers, Statistical mechanics of sequence-dependent circular DNA and its application for DNA cyclization. Biophys. J. 84 (2003) 136-153.
[14] L. Ulanovsky, M. Bodner, E. N. Trifonov, M. Choder, Curved DNA: design, synthesis, and circularization. PNAS 83 (1986) 862-866.
[15] D. Shore, J. Langowski, R. L. Baldwin, DNA flexibility studied by covalent closure of short fragments into circles. PNAS 78 (1981) 4833-4837.
[16] B. Révet, A. Fourcade, Short unligated sticky ends enable the observation of circularised DNA by atomic force and electron microscopies. Nucleic Acids Res. 26 (1998) 2092-2097.
[17] A. Dhanoya, B. M. Chain, E. Keshavarz-Moore, The impact of DNA topology on polyplex uptake and transfection efficiency in mammalian cells. J. Biotech 155 (2011) 377-386.
[18] P. Chancham, J. A. Hughes, Relationship between plasmid DNA topological forms and in vitro transfection. J. Lipos. Res. 11 (2001) 139-152.
[19] V. Gotea, A. Visel, J. M. Westlund, M. A. Nobrega, L. A. Pennacchio, I. Ovcharenko, Homotypic clusters of transcription factor binding sites are a key component of human promoters and enhancers. Genome Res. 20 (2010) 565.
[20] T. H. Lee, N. Maheshri, A regulatory role for repeated decoy transcription factor binding sites in target gene expression. Mol. Syst. Biol. 8 (2012) 576.

1. The method for regulating recombinant gene expression in vitro comprising:
a) providing a mammalian host cell encoding one or more recombinant genes for expression,
b) contacting the cell with a exonuclease resistant block-decoy molecule under conditions suitable for the block-decoy to gain entry into the cell, wherein 2 to 30 regulator element blocks are employed in the block decoy molecule and the regulator element blocks are specific to a transcription factor independently selected from NFkB, CREB, c-Myc, Activator protein 1, CCAAT-enhancer Binding Protein α, Cellular myeloblastosis protein, Elongation Factor 2, Early Growth Response Protein 1, ERR-alpha, GATA-1, AGATAG; Growth Factor Independence 1, Hepatocyte Nuclear Factor 1α, Insulin Promoter Factor 1, IFN-stimulated gene factor 3, Myocyte enhancer Factor 2, Nuclear Factor 1, Nuclear Factor of Activated T Cells, Octamer-1, Retinoic Acid Receptor α, Specificity Protein 1, and Yin Yang 1, and
c) expressing the recombinant protein or proteins.

2. The method according to claim 1, which further comprises recovering the protein or proteins.

3. The method according to claim 1, wherein the mammalian cell is a CHO cell.

4. (canceled)

5. The method according to claim 1, wherein 7 to 27 regulatory element blocks are employed in the block-decoy molecule.

6. (canceled)

7. (canceled)

8. The method according to claim 1, wherein the block-decoy molecule is chimeric.

9. The method according to claim 1, wherein the block decoy is deoxyribonucleic acid.

10. The method according to claim 9, wherein the deoxyribonucleic acid is circular in form.

11. (canceled)

12. The method according to claim 1, wherein part of all of the deoxyribonucleic acid is double stranded.

13. The method of preparing a circular double stranded block-decoy molecule resistant to exonucleases comprising:
a.) forming doubled stranded regulatory element blocks comprising a specific transcription factor binding site with sticky ends by annealing complementary single stranded oligodeoxyribonucleic acids containing a motif to form said transcription factor binding site,
b) ligating the double stranded regulator element-blocks formed in step a) to form a concatemer, and
c) circularisation and ligation of the termini of concatemer formed in step b) to provide a circular block-decoy molecule comprising in the range of 2 and 30 regulatory element-blocks specific to a transcription factor independently selected from NFkB, CREB, c-Myc, Activator protein 1, CCAAT-enhancer Binding Protein α, Cellular myeloblastosis protein, Elongation Factor 2, Early Growth Response Protein 1, ERR-alpha, GATA-1, AGATAG, Growth Factor Independence 1, Hepatocyte Nuclear Factor 1α, Insulin Promoter Factor 1, IFN-stimulated gene factor 3, Myocyte enhancer Factor 2, Nuclear Factor 1, Nuclear Factor of Activated T Cells, Octamer-1, Retinoic Acid Receptor α, Specificity Protein 1, and Yin Yang 1.

14. (canceled)

15. (canceled)

16. The method according to any one of claim 13, wherein the sticky end is overhanging base pairs at the 5′ end or 3′ end, for example at the 5′ end.

17. The method according to claim 16, wherein the overhang is 3 to 10 base pairs.

18. The method according to claim 1, wherein oligodeoxyribonucleic acid in double stranded regulator element-blocks of b) is phosphorylated and capable forming phosphate ester linkages.

19. (canceled)

20. The method according to any one of claim 13, wherein the annealing is performed by denaturation at an elevated temperature, for example 90° C. or above, followed by ramp cooling at a rate between 0.5-1.5° C./minute.

21. (canceled)

22. (canceled)

23. The method according to claim 13, wherein the concatemer is in the range of about 90 to 350 base pairs in length.

24. (canceled)

25. A circular double stranded block-decoy molecule comprising two or more regulator element-blocks in tandem wherein the molecule comprises in the range of 2 to 30 regulatory element blocks specific to a transcription factor independently selected from NFkB, CREB, c-Myc, Activator protein 1, CCAAT-enhancer Binding Protein α, Cellular myeloblastosis protein, Elongation Factor 2, Early Growth Response Protein 1, ERR-alpha, GATA-1, AGATAG, Growth Factor Independence 1, Hepatocyte Nuclear Factor 1α, Insulin Promoter Factor 1, IFN-stimulated gene factor 3, Myocyte enhancer Factor 2, Nuclear Factor 1, Nuclear Factor of Activated T Cells, Octamer-1, Retinoic Acid Receptor α, Specificity Protein 1, and Yin Yang 1.

26. (canceled)

27. (canceled)

28. The circular double stranded molecular according claim 25, wherein the molecule is deoxyribonucleic acid.

29. The circular double stranded molecule according to claim 28, comprising 100 base pairs or more.

30. The circular double stranded molecule according claim 25, which is exonuclease resistant.

31. (canceled)

32. A library of block-decoy molecules comprising a plurality of molecules as defined in claim 25, wherein the block decoy molecules in the library have different levels, ratios and/or combinations or regulatory element blocks.

33. The library according to claim 32, comprising 100 or more molecules.

34. (canceled)

35. (canceled)


SEQUENCE LISTING (25 sequences)

SEQ ID NO: 1 - DNA, 16 bp, Artificial
  Regulatory element
tcgatgggactttcca

SEQ ID NO: 2 - DNA, 16 bp, Artificial
  Regulatory Element
tcgatggaaagtccca

SEQ ID NO: 3 - DNA, 16 bp, Artificial
  Regulatory Element
tcgatttgacgtcatt

SEQ ID NO: 4 - DNA, 16 bp, Artificial
  Regulatory Element
tcgaaatgacgtcaaa

SEQ ID NO: 5 - DNA, 15 bp, Artificial
  Regulatory Element
tcgaaacacgtgaga

SEQ ID NO: 6 - DNA, 15 bp, Artificial
  Regulatory Element
tcgatctcacgtgtt

SEQ ID NO: 7 - DNA, 15 bp, Artificial
  Regulatory Element
aggtcattttgacct

SEQ ID NO: 8 - DNA, 13 bp, Artificial
  Regulatory Element
gggccaaaggtct

SEQ ID NO: 9 - DNA, 12 bp, Artificial
  Regulatory Element
cccattagggac

SEQ ID NO: 10 - DNA, 13 bp, Artificial
  Regulatory Element
gaaaagtgaaacc

SEQ ID NO: 11 - DNA, 10 bp, Artificial
  Regulatory Element
ctaaaaatag

SEQ ID NO: 12 - DNA, 15 bp, Artificial
  Regulatory Element
ttggctatatgccaa

SEQ ID NO: 13 - DNA, 17 bp, Artificial
  Regulatory Element
aggtcatcaagaggtca

SEQ ID NO: 14 - DNA, 10 bp, Artificial
  Regulatory Element
ccaaatttgg

SEQ ID NO: 15 - DNA, 10 bp, Artificial
  Regulatory Element
gtgacgtaac

SEQ ID NO: 16 - DNA, 15 bp, Artificial
  Regulatory Element
aggtcattttgacct

SEQ ID NO: 17 - DNA, 15 bp, Artificial
  Regulatory Element
agaacattttgttct

SEQ ID NO: 18 - DNA, 11 bp, Artificial
  Regulatory Element
aatagggactt

SEQ ID NO: 19 - DNA, 13 bp, Artificial
  Regulatory Element
gggccaaaggtct

SEQ ID NO: 20 - DNA, 12 bp, Artificial
  Regulatory Element
cccattagggac

SEQ ID NO: 21 - DNA, 13 bp, Artificial
  Regulatory Element
gaaaagtgaaacc

SEQ ID NO: 22 - DNA, 10 bp, Artificial
  Regulatory Element
ctaaaaatag

SEQ ID NO: 23 - DNA, 15 bp, Artificial
  Regulatory Element
ttggctatatgccaa

SEQ ID NO: 24 - DNA, 10 bp, Artificial
  Regulatory Element
gggactttcc

SEQ ID NO: 25 - DNA, 17 bp, Artificial
  Regulatory Element
aggtcatcaagaggtca